(19) United States
(12) Patent Application Publication
(10) Pub. No.: US 2016/0267659 A1
(43) Pub. Date: Sep. 15, 2016

(54) METHOD AND DEVICE FOR CO-REGISTERING A MEDICAL 3D IMAGE AND A SPATIAL REFERENCE

(71) Applicant: Brainlab AG, Feldkirchen (DE)

(72) Inventors: Brian VASEY, Munich (DE); Rupert HEIGL, Markt Schwaben (DE); Hubert GOETTE, Feldkirchen (DE)

(21) Appl. No.: 15/031,390

(22) PCT Filed: Dec. 11, 2013
(86) PCT No.: PCT/EP2013/076149

§ 371 Date: Apr. 22, 2016

(30) Foreign Application Priority Data
Oct. 25, 2013 (EP) PCT/EP2013/072430
Oct. 25, 2013 (EP) PCT/EP2013/072434

(51) Int. Cl. G06T 7/00

(52) U.S. Cl. CPC G06T 7/0024; G06T 7/0012; A61B 2090/3954

(57) ABSTRACT

A data processing method for co-registering a medical 3D image dataset and a spatial reference (27), comprising the steps of: acquiring the 3D image dataset, wherein the 3D image dataset represents a medical CT image, a medical MR image or an angiograph of at least a part (20) of a patient and a set of hybrid markers (11); detecting the positions of the hybrid markers (11) in the 3D image dataset so as to obtain a scan matrix representing the arrangement of the set of hybrid markers (11) in the 3D image dataset and the position of the scan matrix in the 3D image dataset; acquiring the positions of the hybrid markers (11) with respect to the spatial reference (27), so as to obtain an image matrix (22a) representing the arrangement of the set of hybrid markers (11) in three-dimensional space and the position of the image matrix (22a) relative to the spatial reference (27); and co-registering the scan matrix and the image matrix (22a).

The present invention relates to a data processing method and a device for co-registering a medical 3D image dataset and a spatial reference.

Using medical navigation systems in order to track and/or navigate medical instruments or other objects has become increasingly popular. In addition, it is common to obtain three-dimensional medical image data using imaging modalities such as computed tomography (CT), magnetic resonance (MR) or angiography. In order to combine the advantages of medical navigation and 3D images, it is necessary to co-register the 3D image and a spatial reference, such as a reference of the medical navigation system. This is typically achieved using markers.

In one common approach, fiducial markers are installed on the patient. Fiducial markers are markers which are easily recognisable in the 3D image dataset. Bone fiducials are screwed directly into a bone. They offer very precise localisation, but are invasive. Non-invasive markers use an adhesive to hold the markers on the patient. A first conventional approach localises the fiducial markers in the 3D image dataset. The position of the fiducial markers of the reference of the medical navigation system is then determined by sampling the markers using an instrument such as a pointer. The locations of the fiducial markers in the 3D image dataset and the sampled locations can then be co-registered.

In another conventional approach, a device comprising different types of markers is used. This device comprises an array of fiducial markers in a known arrangement, i.e. having known locations relative to each other. The device also comprises an array of markers which can be detected by the medical navigation system. Such markers are in particular optical markers, in particular infrared-reflective (IR-reflective) markers. The relative position between the two arrays is known, such that the 3D image dataset and the reference of the medical navigation system can be co-registered once the locations of the fiducial markers in the 3D image dataset and the positions of the navigation system markers of the reference of the navigation system have been determined.

As outlined above, bone fiducials require an invasive procedure in order to be installed. Non-invasive markers introduce the problem of skin shift. Since the skin is pliable, the marker can move while it is being sampled. During registration, the pointer has to be placed in the centre of the fiducial marker in order to accurately record its location. This can mean applying a downward or lateral pressure to the marker. If the marker is located in an area of loose skin, this can cause the marker to move, thus creating an error in its sampled location.

A device comprising two different types of markers requires the relative position between the two different arrays of markers to he known and precisely maintained. This necessitates a very rigid and therefore complex and/or heavy structure to bear the markers.

The inventors have found that using hybrid medical markers can facilitate co-registration. Such hybrid medical markers can be detected both in the medical 3D image dataset and by the medical navigation system. Hybrid medical markers can also be referred to simply as hybrid markers. A set of hybrid markers can also be referred to as a matrix of hybrid markers, a marker matrix or simply a matrix, wherein the term “matrix” does not limit the set of hybrid markers to a regular arrangement. Any invariant arrangement of hybrid markers within a set of hybrid markers is possible, such that the hybrid markers (of the set of hybrid markers) have a known and fixed relative position in three-dimensional space. The marker matrix must be invariant, i.e. its markers must exhibit a fixed relative position relative to each other, at the time the markers are detected in three-dimensional space and the medical 3D image dataset is recorded.

The present invention relates to a method for co-registering a medical 3D image dataset and a spatial reference. The method comprises the steps of: acquiring the 3D image dataset, wherein the 3D image dataset represents a medical CT image, a medical MR image or an angiograph of at least a part of a patient and a set of hybrid markers; detecting the positions of the hybrid markers in the 3D image dataset so as to obtain a scan matrix representing the arrangement of the set of hybrid markers in the 3D image dataset; acquiring the positions of the hybrid markers with respect to the spatial reference, so as to obtain an image matrix representing the arrangement of a set of hybrid markers in three-dimensional space; and co-registering the scan matrix and the image matrix.

The 3D image dataset represents a three-dimensional medical image of a particular spatial region in which at least a part of the patient and the hybrid markers are located. Devices and methods for obtaining CT images, MR images or angiographs are known in the art. The hybrid markers have properties such that they are visible in the three-dimensional medical image.

Methods and algorithms for detecting markers in a 3D image dataset are known, in particular if properties such as the size or material of the markers are known. Once they have been detected, the positions of the hybrid markers in the set of hybrid markers in the 3D image dataset, and therefore the arrangement of the hybrid markers in the 3D image dataset, is known. However, the positions are defined with respect to the 3D image dataset only and not with respect to the spatial reference.

In this document, the term “position” of an object means the spatial location of the object in up to three translational dimensions and/or the rotational alignment of the object in up two three rotational dimensions. For a marker, such as the hybrid markers described above, “position” typically means the spatial location only, since markers are typically symmetrical.

The “arrangement” of the hybrid markers in the 3D image dataset means the relative positions of the hybrid markers in the 3D image dataset. The scan matrix is therefore a virtual representation of the actual matrix in the 3D image dataset.

As outlined above, detecting the position of the scan matrix in the 3D image dataset in particular means determining the position of the scan matrix relative to a reference of the 3D image dataset, which can for example be a co-ordinate system of the 3D image dataset.

The positions of the hybrid markers with respect to the spatial reference can be acquired using known means, such as a medical navigation system. The chosen means for acquiring the positions of the hybrid markers depends on the type of hybrid markers, such as for example an optical hybrid marker which is light-reflective or an electromagnetic hybrid marker which emits electromagnetic radiation.

The arrangement of the set of hybrid markers in three-dimensional space means the relative position of the hybrid markers in three-dimensional space. The means for determining the position of a marker in three-dimensional space is highly accurate, such that the obtained image matrix can be considered to be identical to the marker matrix.

Co-registering the scan matrix and the image matrix means determining a transformation which rotates and optionally shifts the scan matrix such that it matches the image matrix. In other words, the co-registered scan matrix and image matrix are congruent. If the positions of the hybrid markers are correctly detected in the 3D image dataset, then co-registering can optionally only require an additional scaling in order to achieve congruence. If the scan matrix and image matrix cannot be made congruent by shifting, rotating and scaling the scan matrix, then the step of co-registering can optionally involve an elastic fusion which changes the shape of the scan matrix. The obtained transformation is then applied to the 3D image dataset such that the 3D image dataset and the spatial reference are co-registered.

Alternatively, co-registering the scan matrix and the image matrix can involve adapting the 3D image dataset, in particular the whole 3D image dataset including the scan matrix, such that the scan matrix within the 3D image dataset matches the image matrix. The 3D image dataset is then already transformed while the matrices are being matched.

If the 3D image dataset and the spatial reference are co-registered, then the virtual position of the 3D image in three-dimensional space is known. As a result, it is for example possible to show the position of a navigated medical instrument in or relative to the registered 3D image dataset.

The spatial reference can be a reference of the medical navigation system, such as the co-ordinate system of a stereoscopic image captured using a stereoscopic camera. In a preferred embodiment, however, the method also comprises the steps of acquiring the position of a reference marker device and using said position as the spatial reference. In this embodiment, the spatial reference can be located close to the hybrid markers, which increases the registration accuracy.

In one embodiment, acquiring the positions of the hybrid markers with respect to the spatial reference involves acquiring a stereoscopic dataset which represents a 3D optical image of the hybrid markers and detecting the positions of the hybrid markers in the stereoscopic dataset.

The 3D optical image is preferably captured using a stereoscopic camera of a medical navigation system. The 3D optical image is preferably an infrared 3D optical image.

A hybrid medical marker can comprise a marker core which comprises a contrast medium, and an outer surface which is at least partly light-reflective. The contrast medium is a material which can be detected in the 3D image dataset. Since the outer surface of the marker is light-reflective, the marker can be detected by a camera of a medical navigation system.

A medical navigation system typically comprises a stereoscopic camera which captures two images from positions which are spaced apart. Since the distance between the lenses of the stereoscopic camera is known, the position of a marker in the reference of the medical navigation system can be determined from the stereoscopic image. In one common implementation, the stereoscopic camera operates in the infrared spectrum. An infrared light source on or close to the stereoscopic camera emits light which is then reflected by the marker and captured by the stereoscopic camera. In this case, the outer surface of the marker is at least partly light-reflective in the infrared (IR) spectrum. The light-reflective property of the marker's surface is preferably obtained by using one or more retro-reflectors.

The contrast medium is preferably a material which is visible in at least one of the following imaging modalities: x-ray, CT and MR. This ensures that the marker core can be reliably identified in the 3D medical image dataset.

In one embodiment, the marker core consists of a solid contrast medium. In another embodiment, the contrast medium is a liquid, such as a multi-modal hydrogel. In this case, the marker core is preferably a housing which is filled with the liquid contrast medium. The housing is or can be sealed such that the liquid contrast medium cannot leak out of the housing.

If the hybrid medical marker is to be used with a computed tomograph machine, the contrast medium preferably has a Hounsfield unit measurement which is distinct from the Hounsfield unit measurements of the substances which are part of the body, such as fat, water, grey matter or white matter.

As already explained above, at least a part of the outer surface of the hybrid marker is light-reflective. In one embodiment, the outer surface of the marker is at least partly provided with a light-reflective coating. This light-reflective coating is for example a light-reflective paint which is deposited on the outer surface. In another embodiment, the outer surface of the marker is at least partly provided with a light-reflective foil. Such light-reflective foil can comprise a plurality of retro-reflectors, in particular micro-retro-reflectors, which reflect incident light in a parallel manner.

In one embodiment, the marker core has a spherical shape. If a liquid contrast medium is used, then the spherical core is preferably made of two hemispherical shells which are assembled to form a hollow sphere which is filled with the contrast medium. In a preferred embodiment, the whole outer surface of the spherical marker core is light-reflective, with the optional exception of a portion in which a mounting means is provided. Such a spherical hybrid marker can be easily detected by the stereoscopic camera of the medical navigation system, irrespective of its orientation.

Alternatively, the marker core has a cylindrical shape, in which case the marker core is in particular shaped as a flat cylinder or disc, i.e. the height of the cylinder or disc is smaller than its radius.

The cylindrical hybrid marker preferably comprises an adhesive base attached to one of the front faces of the cylindrical core. A front face of the cylindrical core is one of the two flat surfaces of the cylinder. The adhesive base can for example comprise an adhesive tape or Velcro fastening. The hybrid marker can easily be attached to an object via its adhesive base.

In a preferred embodiment, only one of the front faces of the cylindrical core of the hybrid marker is light-reflective, i.e. the hybrid marker has one circular light-reflective portion. It is then simple for the medical navigation system to determine the centre of this circular area from the reflected light captured.

In one embodiment of the invention, the method also comprises the step of adapting the detected positions of the hybrid markers in the 3D image dataset if the hybrid markers have a cylindrical shape. This is particularly useful if only one front face of the cylinder is light-reflective. In this case, a medical navigation system determines the centre of the light-reflective front face of the cylindrical marker as the position of the hybrid marker in three-dimensional space. However, the position of the hybrid marker in the 3D image dataset is typically the position of the centre of the cylinder. Two different points of the cylindrical marker therefore correspond to the position of the cylindrical marker in the 3D image dataset and in three-dimensional space, respectively, contrary to the equivalent scenario when using spherical markers. It is therefore advantageous to adapt the detected position of cylindrical hybrid markers in the 3D image dataset.

In one embodiment, adapting a detected position involves determining the axial direction of the cylindrical hybrid marker and shifting the detected position along this axial direction by half the height of the cylinder. The corrected position of a hybrid marker in the 3D image dataset is then the position of the centre of the light-reflective front face. The axial direction of the cylindrical hybrid marker is preferably determined from the 3D image dataset. A cylindrical hybrid marker is actually represented by a cylindrical object in the 3D image dataset, such that the size and orientation of the cylindrical marker can be determined in the 3D image dataset.

In one embodiment, the method involves repeating the step of detecting the positions of the hybrid markers in the 3D image dataset for different search parameters, so as to obtain a plurality of candidate scan matrices. In this embodiment, the method also comprises the additional steps of co-registering each of the candidate scan matrices and the image matrix and selecting the candidate scan matrix which best matches the image matrix as the scan matrix.

The search parameters can comprise a marker diameter and/or a marker shape and/or a matrix material threshold and/or other parameters. The marker diameter defines the size of a hybrid marker. The marker shape defines the design of the hybrid marker, for example whether it has a spherical or cylindrical design. The matrix material threshold is a threshold value for determining whether or not a voxel in the 3D image dataset is considered to belong to a marker or not. This threshold depends on the imaging modality and the material of the marker.

This embodiment addresses the problem of imperfect marker detection in the 3D image dataset, which in particular arises if the apparatus used for obtaining the 3D image dataset was not properly calibrated. In this embodiment, a plurality of candidate scan matrices are obtained for different search parameters, while the image matrix is preferably considered to correctly represent the actual marker matrix. The candidate scan matrix which best matches the image matrix is then selected as the scan matrix, because the corresponding search parameters are considered to produce the best detection result when detecting the positions of the hybrid markers in the 3D image dataset.

The invention also relates to a program which, when running on a computer, causes the computer to perform one or more or all of the method steps described herein and/or to a program storage medium on which the program is stored (in particular in a non-transitory form) and/or to a computer on which the program is running or into the memory of which the program is loaded and/or to a signal wave, in particular a digital signal wave, carrying information which represents the program, in particular the aforementioned program, which in particular comprises code means which are adapted to perform any or all of the method steps described herein.

The invention also relates to a device for co-registering a medical 3D image dataset and a spatial reference, comprising a computer onto which the aforementioned program is loaded. The device is preferably a medical navigation system for computer-assisted surgery.

Within the framework of the invention, computer program elements can be embodied by hardware and/or software (this includes firmware, resident software, micro-code, etc.). Within the framework of the invention, computer program elements can take the form of a computer program product which can be embodied by a computer-usable, in particular computer-readable data storage medium comprising computer-usable, in particular computer-readable program instructions, “code” or a “computer program” embodied in said data storage medium for use on or in connection with the instruction-executing system. Such a system can be a computer; a computer can be a data processing device comprising means for executing the computer program elements and/or the program in accordance with the invention, in particular a data processing device comprising a digital processor (central processing unit or CPU) which executes the computer program elements, and optionally a volatile memory (in particular a random access memory or RAM) for storing data used for and/or produced by executing the computer program elements. Within the framework of the present invention, a computer-usable, in particular computer-readable data storage medium can be any data storage medium which can include, store, communicate, propagate or transport the program for use on or in connection with the instruction-executing system, apparatus or device. The computer-usable, in particular computer-readable data storage medium can for example be, but is not limited to, an electronic, magnetic, optical, electromagnetic, infrared or semiconductor system, apparatus or device or a medium of propagation such as for example the Internet. The computer-usable or computer-readable data storage medium could even for example be paper or another suitable medium onto which the program is printed, since the program could be electronically captured, for example by optically scanning the paper or other suitable medium, and then compiled, interpreted or otherwise processed in a suitable manner. The data storage medium is preferably a non-volatile data storage medium. The computer program product and any software and/or hardware described here form the various means for performing the functions of the invention in the example embodiments. The computer and/or data processing device can in particular include a guidance information device which includes means for outputting guidance information. The guidance information can be outputted, for example to a user, visually by a visual indicating means (for example, a monitor and/or a lamp) and/or acoustically by an acoustic indicating means (for example, a loudspeaker and/or a digital speech output device) and/or tactilely by a tactile indicating means (for example, a vibrating element or a vibration element incorporated into an instrument). For the purpose of this document, a computer is a technical computer which in particular comprises technical, in particular tangible components, in particular mechanical and/or electronic components. Any device mentioned as such in this document is a technical and in particular tangible device.

It is the function of a marker to be detected by a marker detection device (for example, a camera or an ultrasound receiver or analytical devices such as CT or MRI) in such a way that its spatial position (i.e. its spatial location and/or alignment) can be ascertained. The detection device is in particular part of a navigation system. The markers can be active markers. An active marker can for example emit electromagnetic radiation and/or waves which can be in the infrared, visible and/or ultraviolet spectral range. The marker can also however be passive, i.e. can for example reflect electromagnetic radiation in the infrared, visible and/or ultraviolet spectral range or can block x-ray radiation. To this end, the marker can be provided with a surface which has corresponding reflective properties or can be made of metal in order to block the x-ray radiation. It is also possible for a marker to reflect and/or emit electromagnetic radiation and/or waves in the radio frequency range or at ultrasound wavelengths. A marker preferably has a spherical and/or spheroid shape and can therefore be referred to as a marker sphere; markers can however also exhibit a cornered, for example cubic, shape.

A marker device can for example be a reference star or a pointer or a single marker or a plurality of (individual) markers which are then preferably in a predetermined spatial relationship. A marker device comprises one, two, three or more markers, wherein two or more such markers are in a predetermined spatial relationship. This predetermined spatial relationship is in particular known to a navigation system and is for example stored in a computer of the navigation system.

A “reference star” refers to a device with a number of markers, advantageously three, four or more markers, attached to it, wherein the markers are (in particular detachably) attached to the reference star such that they are stationary, thus providing a known (and advantageously fixed) position of the markers relative to each other. The position of the markers relative to each other can be individually different for each reference star used within the framework of a surgical navigation method, in order to enable a surgical navigation system to identify the corresponding reference star on the basis of the position of its markers relative to each other. It is therefore also then possible for the objects (for example, instruments and/or parts of a body) to which the reference star is attached to be identified and/or differentiated accordingly. In a surgical navigation method, the reference star serves to attach a plurality of markers to an object (for example, a bone or a medical instrument) in order to be able to detect the position of the object (i.e. its spatial location and/or alignment). Such a reference star in particular features a way of being attached to the object (for example, a clamp and/or a thread) and/or a holding element which ensures a distance between the markers and the object (in particular in order to assist the visibility of the markers to a marker detection device) and/or marker holders which are mechanically connected to the holding element and which the markers can be attached to.

A navigation system, in particular a surgical navigation system, is understood to mean a system which can comprise; at least one marker device; a transmitter which emits electromagnetic waves and/or radiation and/or ultrasound waves; a receiver which receives electromagnetic waves and/or radiation and/or ultrasound waves; and an electronic data processing device which is connected to the receiver and/or the transmitter, wherein the data processing device (for example, a computer) in particular comprises a processor (CPU) and a working memory and advantageously an indicating device for issuing an indication signal (for example, a visual indicating device such as a monitor and/or an audio indicating device such as a loudspeaker and/or a tactile indicating device such as a vibrator) and a permanent data memory, wherein the data processing device processes navigation data forwarded to it by the receiver and can advantageously output guidance information to a user via the indicating device. The navigation data can be stored in the permanent data memory and for example compared with data stored in said memory beforehand.

The method in accordance with the invention is in particular a data processing method. The data processing method is preferably performed using technical means, in particular a computer. The data processing method is preferably constituted to be executed by or on a computer and in particular is executed by or on the computer. In particular, all the steps or merely some of the steps (i.e. less than the total number of steps) of the method in accordance with the invention can be executed by a computer. The computer in particular comprises a processor and a memory in order to process the data, in particular electronically and/or optically. The calculating steps described are in particular performed by a computer. Determining steps or calculating steps are in particular steps of determining data within the framework of the technical data processing method, in particular within the framework of a program. A computer is in particular any kind of data processing device, in particular electronic data processing device. A computer can be a device which is generally thought of as such, for example desktop PCs, notebooks, netbooks, etc., but can also be any programmable apparatus, such as for example a mobile phone or an embedded processor. A computer can in particular comprise a system (network) of “sub-computers”, wherein each sub-computer represents a computer in its own right. The term “computer” includes a cloud computer, in particular a cloud server. The term “cloud computer” includes a cloud computer system which in particular comprises a system of at least one cloud computer and in particular a plurality of operatively interconnected cloud computers such as a server farm. Such a cloud computer is preferably connected to a wide area network such as the world wide web (WWW) and located in a so-called cloud of computers which are all connected to the world wide web. Such an infrastructure is used for “cloud computing”, which describes computation, software, data access and storage services which do not require the end user to know the physical location and/or configuration of the computer delivering a specific service. In particular, the term “cloud” is used in this respect as a metaphor for the Internet (world wide web). In particular, the cloud provides computing infrastructure as a service (IaaS). The cloud computer can function as a virtual host for an operating system and/or data processing application which is used to execute the method of the invention. The cloud computer is for example an elastic compute cloud (EC2) as provided by Amazon Web Services™. A computer in particular comprises interfaces in order to receive or output data and/or perform an analogue-to-digital conversion. The data are in particular data which represent physical properties and/or which are generated from technical signals. The technical signals are in particular generated by means of (technical) detection devices (such as for example devices for detecting marker devices) and/or (technical) analytical devices (such as for example devices for performing imaging methods), wherein the technical signals are in particular electrical or optical signals. The technical signals in particular represent the data received or outputted by the computer. The computer is preferably operatively coupled to a display device which allows information outputted by the computer to be displayed, for example to a user. One example of a display device is an augmented reality device (also referred to as augmented reality glasses) which can be used as “goggles” for navigating. A specific example of such augmented reality glasses is Google Glass (a trademark of Google, Inc.). An augmented reality device can be used both to input information into the computer by user interaction and to display information outputted by the computer.

The expression “acquiring data” in particular encompasses (within the framework of a data processing method) the scenario in which the data are determined by the data processing method or program. Determining data in particular encompasses measuring physical quantities and transforming the measured values into data, in particular digital data, and/or computing the data by means of a computer and in particular within the framework of the method in accordance with the invention. The meaning of “acquiring data” also in particular encompasses the scenario in which the data are received or retrieved by the data processing method or program, for example from another program, a previous method step or a data storage medium, in particular for further processing by the data processing method or program. The expression “acquiring data” can therefore also for example mean waiting to receive data and/or receiving the data. The received data can for example be inputted via an interface. The expression “acquiring data” can also mean that the data processing method or program performs steps in order to (actively) receive or retrieve the data from a data source, for instance a data storage medium (such as for example a ROM, RAM, database, hard drive, etc.), or via the interface (for instance, from another computer or a network). The data can be made “ready for use” by performing an additional step before the acquiring step. In accordance with this additional step, the data are generated in order to be acquired. The data are in particular detected or captured (for example by an analytical device). Alternatively or additionally, the data are inputted in accordance with the additional step, for instance via interfaces. The data generated can in particular be inputted (for instance into the computer). In accordance with the additional step (which precedes the acquiring step), the data can also be provided by performing the additional step of storing the data in a data storage medium (such as for example a ROM, RAM, CD and/or hard drive), such that they are ready for use within the framework of the method or program in accordance with the invention. The step of “acquiring data” can therefore also involve commanding a device to obtain and/or provide the data to be acquired. In particular, the acquiring step does not involve an invasive step which would represent a substantial physical interference with the body, requiring professional medical expertise to be carried out and entailing a substantial health risk even when carried out with the required professional care and expertise. In particular, the step of acquiring data, in particular determining data, does not involve a surgical step and in particular does not involve a step of treating a human or animal body using surgery or therapy. In order to distinguish the different data used by the present method, the data are denoted (i.e. referred to) as “XY data” and the like and are defined in terms of the information which they describe, which is then preferably referred to as “XY information” and the like.

It is within the scope of the present invention to combine one or more features of one or more embodiments in order to form a new embodiment wherever this is technically expedient and/or feasible. Specifically, a feature of one embodiment which has the same or a similar function to another feature of another embodiment can be exchanged with said other feature, and a feature of one embodiment which adds an additional function to another embodiment can in particular be added to said other embodiment.

The present invention shall now be explained in more detail with reference to the accompanying drawings, which show:

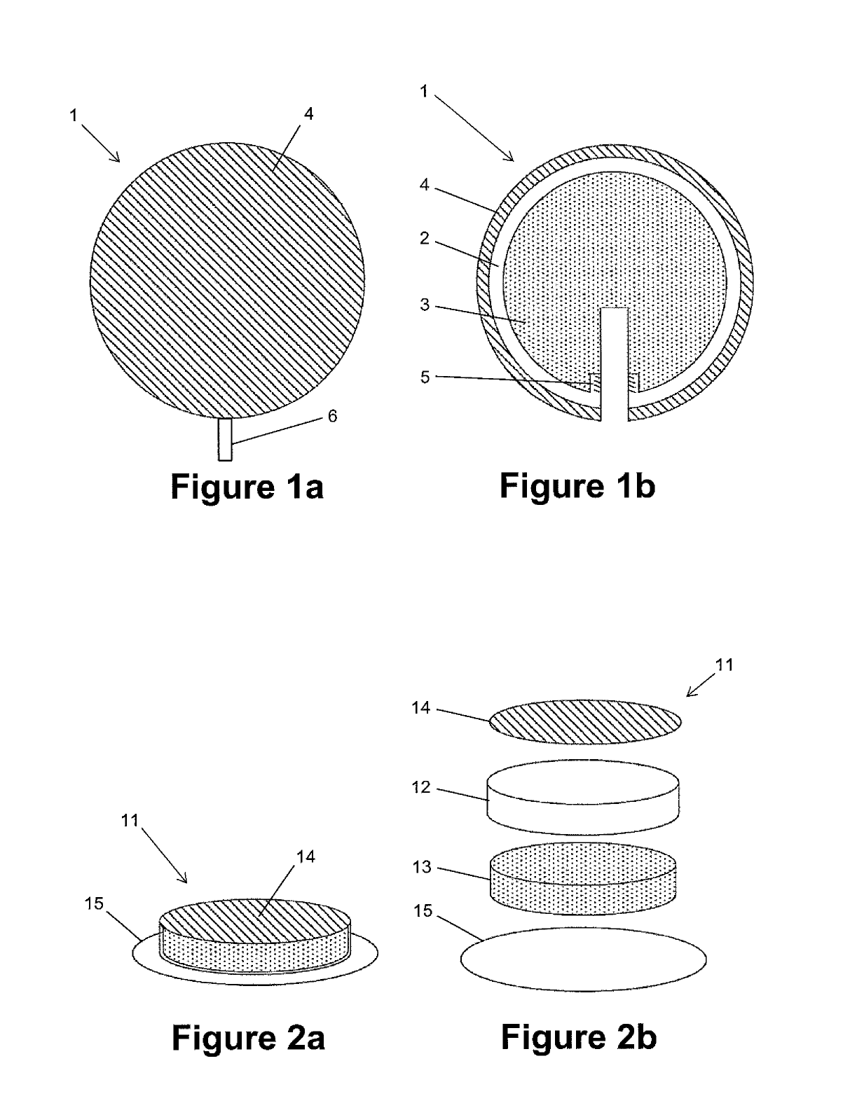

FIG. 1a an external view of a spherical hybrid marker;

FIG. 1b a sectional view of the hybrid marker of FIG. 1a;

FIG. 2a a perspective view of a flat hybrid marker;

FIG. 2b an exploded view of the hybrid marker of FIG. 2a;

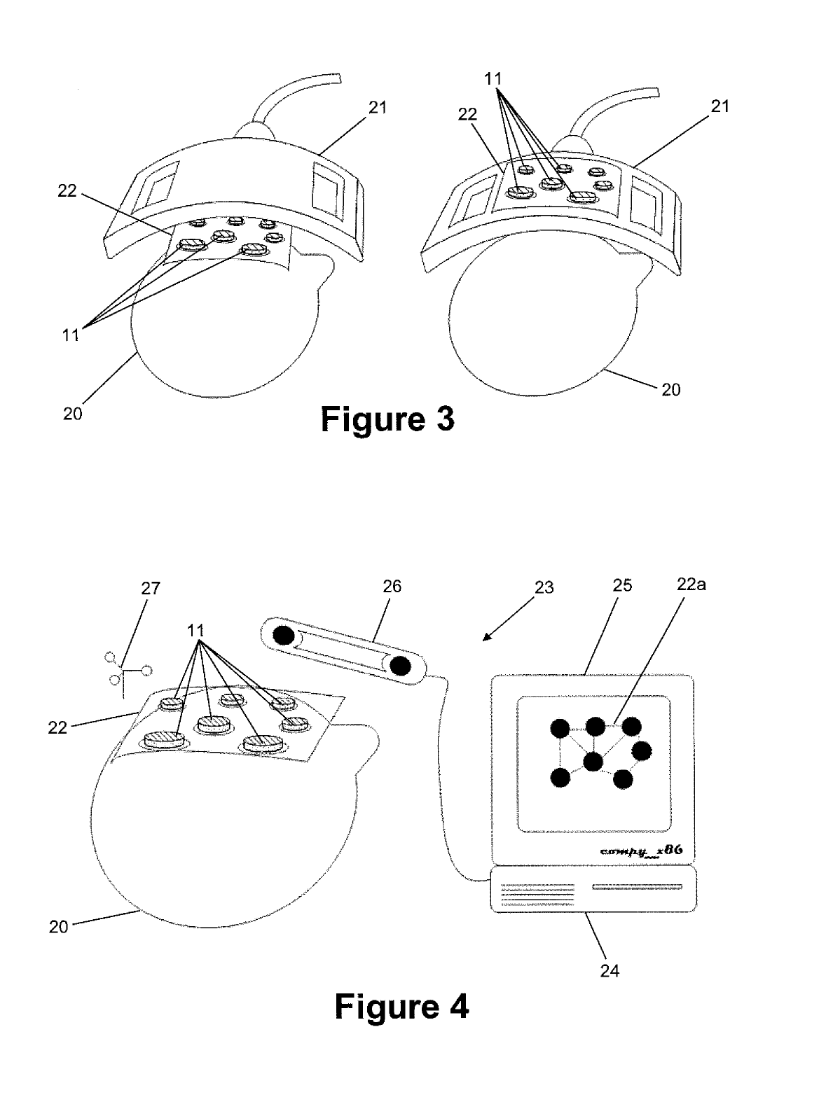

FIG. 3 a marker matrix which is to be detected by an MR scanner;

FIG. 4 the marker matrix of FIG. 3, wherein the matrix is to be detected by a medical navigation system;

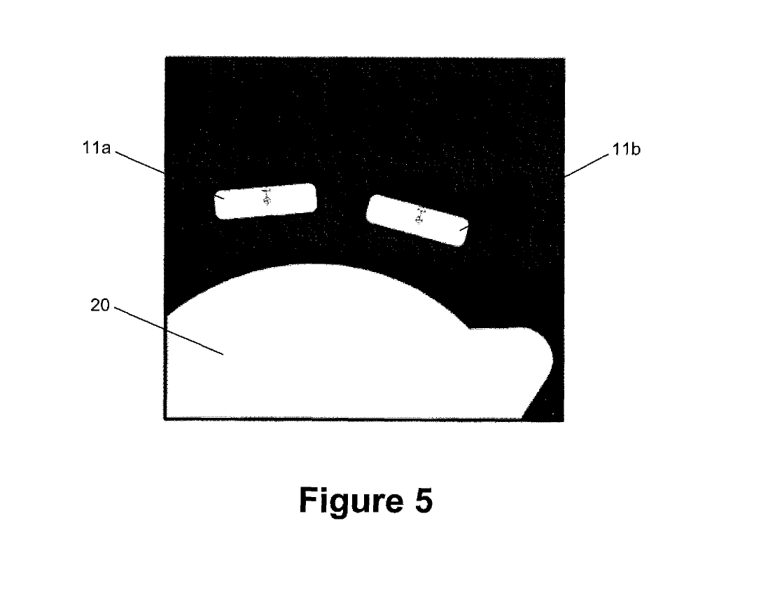

FIG. 5 a sectional 2D image extracted from a 3D image dataset;

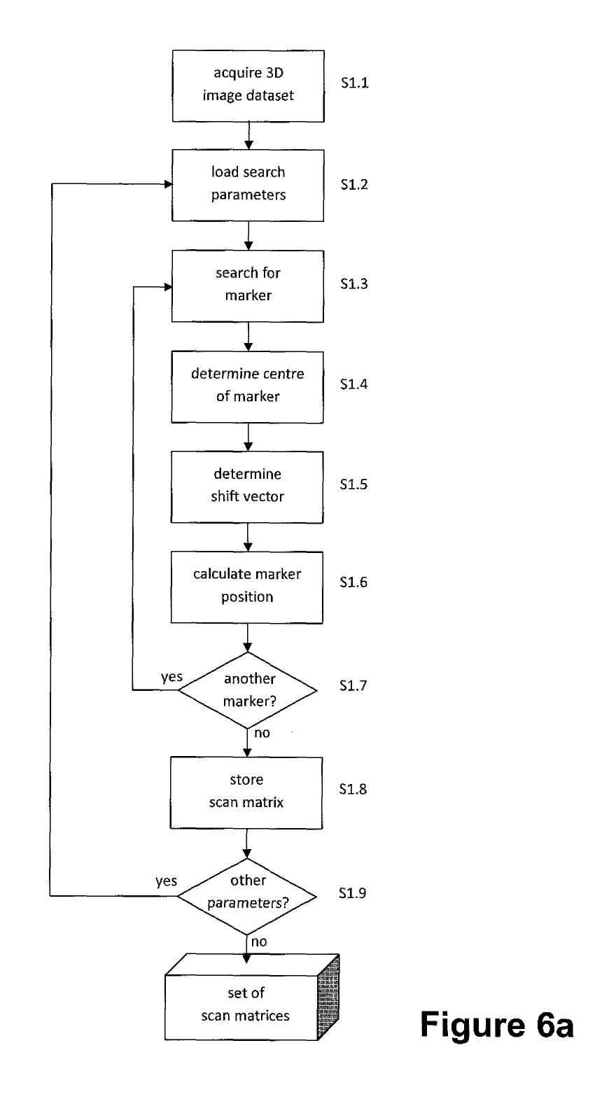

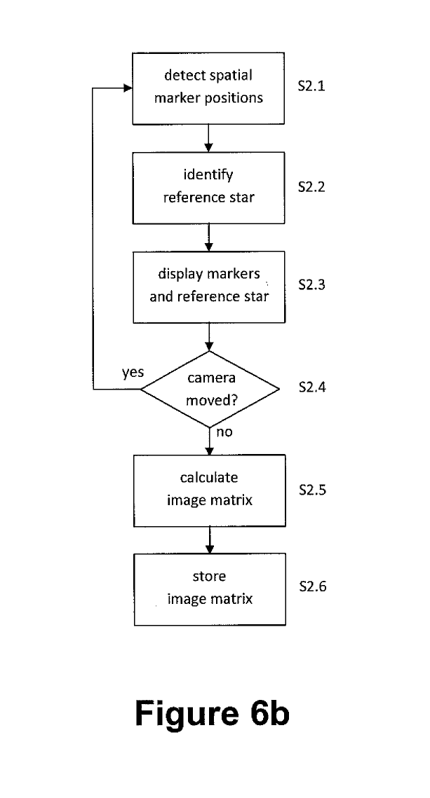

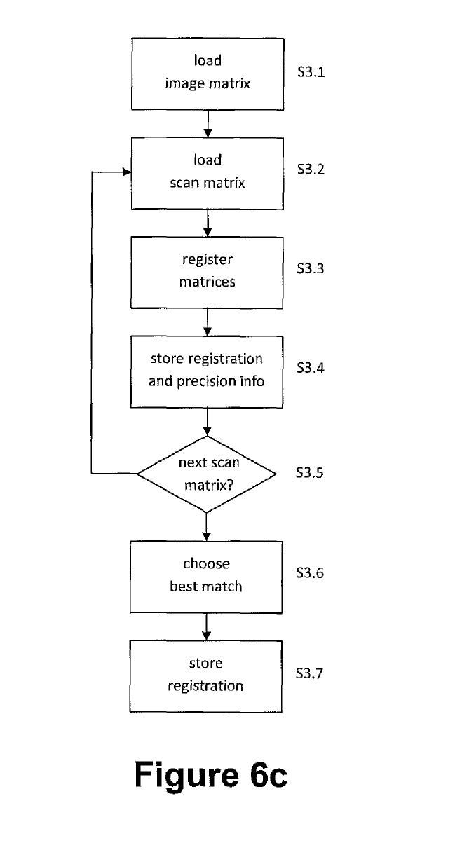

FIG. 6a a flow diagram for obtaining scan matrices;

FIG. 6b a flow diagram for obtaining an image matrix;

FIG. 6c a flow diagram for co-registering the scan matrices and the image matrix;

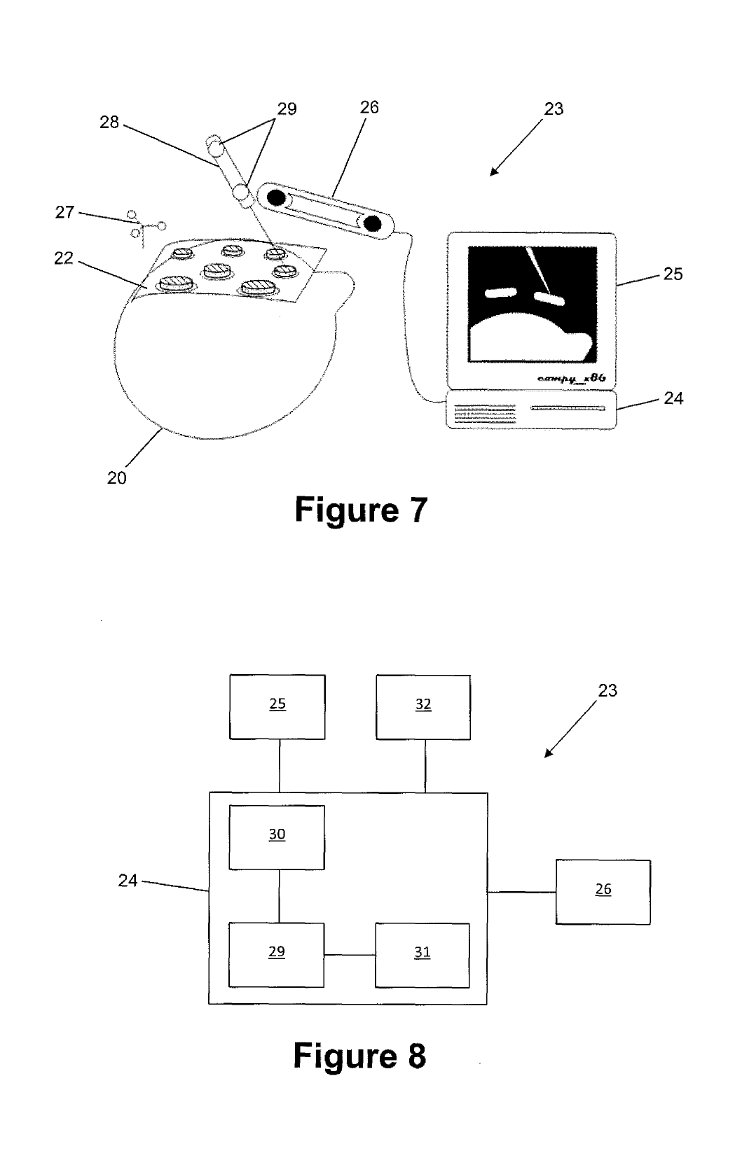

FIG. 7 an environment for navigating a medical instrument relative to the 3D image dataset; and

FIG. 8 a computer for carrying out the invention.

FIG. 1a shows a spherical hybrid medical marker 1, the outer spherical surface of which is covered with a retro-reflective foil 4. The retro-reflective foil 4 reflects light back along its path of incidence. In FIG. 1a, the marker 1 is attached to a pole 6, via which the marker 1 can be attached to an object.

FIG. 1b shows a sectional view of the marker 1 of FIG. 1a. The hybrid marker 1 comprises a core consisting of a spherical housing 2 which is made of plastic and filled with a contrast medium 3. The contrast medium 3 is a material which is visible in a CT or MR image, and for example has a known x-ray attenuation per unit volume, such that it generates a predetermined grey value in a CT image. If the hybrid marker 1 is to be used with an MR imaging apparatus, then the contrast medium 3 is a material which exhibits known MR properties.

As can be seen from FIG. 1b, the housing 2 has an opening 5 comprising a female thread for receiving the mounting pole 6. When the mounting pole 6 is not inserted, which is the state shown in FIG. 1b, a liquid contrast medium 3 can be introduced into or removed from the housing 2. The opening 5 is sealed by the inserted mounting pole 6 in order to prevent the contrast medium 3 from leaking out of the marker 1. The contrast medium 3 can also be a solid contrast medium.

The whole outer surface of the housing 2, except for the region of the opening 5, is covered with the reflective foil 4. Given this configuration, the marker 1 can be detected by a stereoscopic camera of a medical navigation system, irrespective of the orientation of the marker I.

FIG. 2a shows a perspective view of a flat hybrid medical marker 11, FIG. 2b shows an exploded view of the flat hybrid medical marker 11.

The core of the marker 11 is formed by a plastic housing 12 filled with a contrast medium 13. As is also the case with the marker 1, the contrast medium 13 of the marker 11 can be a solid contrast medium or a liquid contrast medium. The housing 12 of the marker 11 has a cylindrical shape, wherein the height of the housing 12 is smaller than its radius. The radius of the housing 12 is in particular at least two, three, five or ten times as large as the height of the housing 12.

The cylindrical housing 12 has two circular front faces, one of which is provided with a reflective foil 14. The reflective foil 14 is in particular a retro-reflective foil comprising a plurality of retro-reflectors. The opposite front face of the housing 12 is attached to an adhesive base 15 via which the marker 11 can be attached to an object.

FIG. 3 shows a matrix 22 of flat hybrid markers 11, the head 20 of a patient, and the coil 21 of a magnetic resonance imaging apparatus. By using the coil 21, the MR imaging apparatus can obtain a three-dimensional MR image as a 3D image dataset.

The marker matrix 22 can for example be arranged between the coil 21 and the head 20, as shown in the left-hand illustration of FIG. 3, and can in particular be attached to the head. Alternatively, however, the marker matrix 22 can also be attached to the coil 21, as shown in the right-hand illustration of FIG. 3. The exact location of the marker matrix 22 is irrelevant as long as it is within the field of view of the coil 21, such that the markers 11 are shown in the 3D medical image represented by the 3D image dataset.

Since the positions of the hybrid markers 11 are determined both in physical space and within the 3D image dataset, the arrangement of the hybrid markers 11, i.e. the relative position of the hybrid markers 11, is likewise irrelevant as long as it is identical at the times the marker positions in physical space and the 3D image dataset are obtained.

FIG. 4 shows a medical navigation system 23 comprising a computer 24 which is connected to a display unit 25 and to a stereoscopic camera 26, The field of view of the stereoscopic camera 26 covers the marker matrix 22 and also a reference star 27 as a spatial reference. In this example embodiment, the reference star 27 consists of three spherical markers in a known arrangement.

The stereoscopic camera 26 captures a stereoscopic optical infrared image of the marker matrix 22 and the reference star 27. In particular, the stereoscopic camera 26 captures two different two-dimensional images using two different two-dimensional cameras which are a known distance apart. The computer 24 calculates the positions of the markers 11 of the marker matrix 22 and the positions of the markers of the reference star 27 from the stereoscopic image provided by the stereoscopic camera 26. The positions of the spherical markers of the reference star 27 correspond to the centres of the respective spherical markers, while the positions of the disc-shaped markers 11 correspond to the centres of the circular reflective foils 14 (see FIGS. 2a and 2b). The image matrix 22a detected by the medical navigation system 23 is shown as an image on the display unit 25.

FIG. 5 shows a two-dimensional image which represents a slice of the three-dimensional MR image represented by the 3D image dataset and obtained using the coil 21. The two-dimensional image shows the contour of the patient's head 20 and the contour of two hybrid markers 11a and 11b. For the sake of simplicity, the two-dimensional image is shown in FIG. 5 as a black-and-white image, whereas a medical 3D image is in fact typically a greyscale image.

The computer 24 detects the positions of the hybrid markers 11 in the 3D image dataset so as to obtain a scan matrix which represents the arrangement of the set of hybrid markers 11 in the 3D image dataset and the position of the scan matrix in the 3D image dataset. Depending on the particular algorithm used to obtain the positions of the hybrid markers 11 in the 3D image dataset, this position may correspond to the centre of the volumes of the hybrid markers 11. This centre is shown in FIG. 5 as a circle with crosshairs in the hybrid markers 11a and 11b. However, as outlined above, the image matrix 22a corresponds to the positions of the centres of the circular foils 14 on the front faces of the hybrid markers 11 rather than to the centre of the volumes of the hybrid markers 11. The positions of the hybrid markers 11 in the 3D image dataset are therefore adapted in order to harmonise the scan matrix and the image matrix.

In particular, the vectors shown within the hybrid markers 11a and 11b are calculated. These vectors coincide with the rotary axis of the cylindrical bodies of the hybrid markers 11. The origins of these vectors are the centres of the volumes of the hybrid markers 11, i.e, the centres which correspond to the detected positions of the hybrid markers 11 in the 3D image dataset. The length of the vectors are half the respective thickness of the disc-shaped hybrid markers 11, which is in particular half the height of the cylindrical core 12 (see FIGS. 2a and 2b) of the hybrid markers 11, The respective positions of the hybrid markers 11 in the 3D image dataset are then shifted by this corresponding vector, i.e. are shifted onto the centre of the front face of each of the hybrid markers 11 which is covered with the reflective foil 14. The positions of the hybrid markers 11 in the 3D image dataset and the positions of the hybrid markers 11 in physical space then correspond to the same points on the front faces of the hybrid markers 11.

FIG. 6a shows a flow diagram of a method for obtaining one or more scan matrices. In step S1.1, the 3D image dataset is acquired. Acquiring the 3D image dataset may involve loading the dataset from a storage medium, receiving the dataset via an interface or calculating the dataset from raw data such as the raw data measured using the coil 21.

In step S1.2, search parameters are loaded. The search parameters relate to the properties of the hybrid markers 11 and in particular define the diameter of the hybrid markers 11 and/or the shape of the hybrid markers 11 and/or a detection threshold. The detection threshold is a threshold used to determine whether or not a voxel of the 3D image dataset belongs to a hybrid marker 11. This threshold depends on the properties of the MR imaging apparatus and/or the properties of the contrast medium 13.

In step S1.3, the computer 24 searches for a hybrid marker 11 in the 3D image dataset. This search is conducted on the basis of the search parameters. Algorithms for searching for markers in 3D image datasets are known to the person skilled in the art and are therefore not explained in detail here.

In step S1.4, the centre of the marker found is determined. The result of this determination is in particular the co-ordinates of the centre of the marker in the 3D image dataset.

In step S1.5, a shift vector is determined. This shift vector extends along the axis of rotational symmetry of the hybrid marker found. The length of this vector is half the axial length of the marker found. This length can be determined from the 3D image dataset or taken from the search parameters.

In step S1.6, the position of the marker found is calculated. This position of the marker found is the position of the centre of the marker, shifted by the corresponding shift vector. The position of the marker found therefore corresponds to the centre of the circular front face of a marker 11 which is the face provided with the reflective foil 14.

The direction of the shift vector depends on the imaging geometry. The shift vectors generally point away from the head 20. In the configuration shown in the left-hand illustration of FIG. 3, the shift vectors point towards the coil 21, and in the right-hand illustration of FIG. 3, the shift vectors point away from the coil 21.

In step S1.7, a determination is made as to whether or not another hybrid marker is to be detected in the 3D image dataset. The number of hybrid markers 11 of the marker matrix 22 is preferably known, such that a number of markers equal to the number of hybrid markers 11 in the marker matrix 22 is determined in the 3D image dataset.

If another marker is to be searched for, the method returns to step S1.3. If no additional markers are to be searched for, the method proceeds from step S1.7 to step S1.8 in which the positions of the markers found in the 3D image dataset are stored as a scan matrix.

In step S1.9, a determination is made as to whether or not a new search for markers in the 3D image dataset is to be performed on the basis of other search parameters. If a new search is to be performed, the method returns to step S 1.2 in which the new search parameters are loaded, and a search for markers is then repeated. If no additional search is to be performed, the method ends with step S1.9.

The iteration of steps S1.2 to S1.9, i.e. searching for markers in the 3D image dataset for different search parameters, is optional. Accordingly, the result of the method shown in FIG. 6a will be either a single scan matrix or a plurality of scan matrices, depending on whether or not different search parameters are used.

FIG. 6b shows a flow diagram of a method for obtaining the image matrix 22a. In step S2.1, the computer 24 detects the positions of the hybrid markers 11 with respect to an internal reference of the medical navigation system 23 from the stereoscopic image captured using the stereoscopic camera 26. The marker co-ordinates in this example are given in a reference co-ordinate system of the navigation system 23, in particular a co-ordinate system associated with the stereoscopic camera 26.

In step S2.2, the computer 24 identifies the reference star 27, and therefore the spatial reference, in the stereoscopic image of the stereoscopic camera 26.

In step S2.3, the detected hybrid markers 11 and the reference star 27 are displayed on the display unit 25. On the basis of this display, an operator can assess the visibility of the hybrid markers 11 and the reference star 27. In view of the assessed level of visibility, the stereoscopic camera 26 may be moved in order to increase the visibility.

In step S2.4, a determination is made as to whether or not the stereoscopic camera 26 has been moved. If the camera is determined to have been moved, the method returns to step S2.1 in order to acquire the positions of the hybrid markers 11 and reference star 27 again. If the camera is determined to not have been moved, the method proceeds from step S2.4 to step S2.5 in which the computer 24 calculates the image matrix 22a from the positions of the hybrid markers 11 in physical space. The computer 24 also calculates the position of the image matrix 22a relative to the reference star 27 as the spatial reference.

The result of the method shown in FIG. 6b is an image matrix and its position relative to the spatial reference 27. It should be noted that steps S2.3 and S2.4 are optional.

FIG. 6c shows a flow diagram of a method for co-registering the one or more scan matrices and the image matrix. In step S3.1, the image matrix is loaded. In step S3.2, one of the scan matrices is loaded. In step S3.3, the image matrix and the loaded scan matrix are registered. In this step, the scan matrix is shifted and rotated and optionally scaled such that it optimally matches the image matrix. Precision information, which represents the similarity between the transformed scan matrix and the image matrix, is also calculated. The precision information is in particular the sum of the distances between corresponding pairs of markers in the transformed scan matrix and the image matrix. If the positions of the hybrid markers 11 have been correctly detected in the 3D image dataset, then the sum of these distances will be 0.

In step S3.4, the transformation and the corresponding precision information for the present scan matrix are stored.

In step S3.5, a determination is made as to whether or not another scan matrix exists. If this is the case, the method returns to step S3.2 in which the next scan matrix is then loaded. If there are no more scan matrices, the method proceeds from step S3.5 to step S3.6 in which the scan matrix which best matches the image matrix after transformation is selected from the plurality of scan matrices. The corresponding transformation is then stored as the registration between the corresponding scan matrix and the image matrix.

It should be noted that steps S3.4, S3.5 and S3.6 are redundant if only one scan matrix has been detected. In this case, it is not necessary to calculate the precision information in step S3.3.

In one optional modification, the method shown in FIG. 6c is repeated wherein an elastic registration of the matrices is performed in step S3.3 rather than a rigid registration if the precision information fulfils a particular criterion, for example if the precision information concerning the best match is above a predetermined threshold. In this case, the scan matrix and also the 3D image dataset are deformed in order to match the image matrix.

The method according to the present invention comprises a combination of the three methods shown in FIGS. 6a, 6b and 6c.

FIG. 7 shows the medical navigation system 23, the patient's head 20, the marker matrix 22 and the reference star 27 in a configuration similar to FIG. 4. In addition, a medical instrument 28 comprising at least three marker spheres 29 is provided within the field of view of the stereoscopic camera 26, such that it can be tracked by the medical navigation system 23. The two-dimensional image shown in FIG. 5, without the markings depicting the centres of the hybrid markers and the shift vectors, is shown on the display unit 25. The computer 24 is also provided with the geometry of the medical instrument 28 and in particular with the position of the tip of the medical instrument 28 relative to the marker spheres 29.

Since the 3D image dataset and the reference star as the spatial reference are co-registered, the computer 24 can superimpose an image of at least a part of the medical instrument 28 onto the two-dimensional image from FIG. 5 in order to display the position of the medical instrument 28 relative to the 3D image dataset.

FIG. 8 schematically shows the structure of the medical navigation system 23. The computer 24 of the medical navigation system 23 comprises a central processing unit 29, a memory unit 30 and an interface 31. The computer 24 is connected to the display unit 25, the stereoscopic camera 26 and an input unit 32.

The memory unit 30 stores a computer program which instructs the central processing unit 29 to perform the method according to the present invention. The memory unit 30 can store additional data such as the 3D image dataset, the image matrix and the scan matrices. The computer 24 can acquire data via the interface 32, such as for example the 3D image dataset which is acquired from an MR imaging apparatus. The computer 24 can however also be a part of the MR imaging apparatus and be connected to the coil 21.

1-9. (canceled)

10. A data processing system, comprising a computer having a processor configured to execute a computer-implemented medical processing method for co-registering a medical 3D image dataset and a spatial reference, comprising the steps of:
acquiring the 3D image dataset, wherein the 3D image dataset represents a medical CT image, a medical MR image or an angiograph of at least a part of a patient and a set of hybrid markers;
detecting the positions of the hybrid markers in the 3D image dataset so as to obtain a scan matrix representing the arrangement of the set of hybrid markers in the 3D image dataset and the position of the scan matrix in the 3D image dataset;
acquiring the positions of the hybrid markers with respect to the spatial reference, so as to obtain an image matrix representing the arrangement of the set of hybrid markers in three-dimensional space and the position of the image matrix relative to the spatial reference;
co-registering the scan matrix and the image matrix;
repeating the step of detecting the positions of the hybrid markers in the 3D image dataset for different search parameters, so as to obtain a plurality of candidate scan matrices;
co-registering each of the candidate scan matrices and the image matrix; and
selecting the candidate scan matrix which best matches the image matrix as the scan matrix.

11. A computer-implemented medical data processing method for co-registering a medical 3D image dataset and a spatial reference, the method comprising executing, on a processor of a computer, the steps of:
acquiring, at the processor, the 3D image dataset, wherein the 3D image dataset represents a medical CT image, a medical MR image or an angiograph of at least a part of a patient and a set of hybrid markers;
detecting, by the processor, the positions of the hybrid markers in the 3D image dataset so as to obtain a scan matrix representing the arrangement of the set of hybrid markers in the 3D image dataset and the position of the scan matrix in the 3D image dataset;
acquiring, at the processor, the positions of the hybrid markers with respect to the spatial reference, so as to obtain an image matrix representing the arrangement of the set of hybrid markers in three-dimensional space and the position of the image matrix relative to the spatial reference;
co-registering, by the processor, the scan matrix and the image matrix;
repeating, by the processor, the step of detecting the positions of the hybrid markers in the 3D image dataset for different search parameters, so as to obtain a plurality of candidate scan matrices;
co-registering, by the processor, each of the candidate scan matrices and the image matrix; and
selecting, by the processor, the candidate scan matrix which best matches the image matrix as the scan matrix.

12. The method according to claim 11, further comprising the steps of acquiring the position of a reference marker device and using said position as the spatial reference.

13. The method according to claim 11, wherein acquiring the positions of the hybrid markers with respect to the spatial reference involves acquiring a stereoscopic dataset which represents a 3D optical image of the hybrid markers and detecting the positions of the hybrid markers in the stereoscopic dataset.

14. The method according to claim 11, further comprising the step of adapting the detected positions of the hybrid markers in the 3D image dataset if the hybrid markers have a cylindrical shape.

15. The method according to claim 14, wherein adapting a detected position involves determining the axial direction of the cylindrical hybrid marker and shifting the detected position along the axial direction by half the height of the cylinder.

16. The method according to claim 15, wherein the axial direction of the cylindrical hybrid marker is determined from the 3D image dataset.

17. A non-transitory computer-readable program storage medium storing a program which, when executed on a processor of a computer, causes the processor to perform the method steps of:
acquiring the 3D image dataset, wherein the 3D image dataset represents a medical CT image, a medical MR image or an angiograph of at least a part of a patient and a set of hybrid markers;
detecting the positions of the hybrid markers in the 3D image dataset so as to obtain a scan matrix representing the arrangement of the set of hybrid markers in the 3D image dataset and the position of the scan matrix in the 3D image dataset;
acquiring the positions of the hybrid markers with respect to the spatial reference, so as to obtain an image matrix representing the arrangement of the set of hybrid markers in three-dimensional space and the position of the image matrix relative to the spatial reference;
co-registering the scan matrix and the image matrix;
repeating the step of detecting the positions of the hybrid markers in the 3D image dataset for different search parameters, so as to obtain a plurality of candidate scan matrices;
co-registering each of the candidate scan matrices and the image matrix; and
selecting the candidate scan matrix which best matches the image matrix as the scan matrix.

18. A computer comprising the non-transitory computer-readable storage medium of claim 17.

19. A device for co-registering a medical 3D image dataset and a spatial reference, comprising the computer according to claim 18.
